(19) United States
(12) Patent Application Publication
(10) Pub. No.: US 2015/0257938 A1
(43) Pub. Date: Sep. 17, 2015

(54) System and Method for Protecting a Wound on the Skin

(71) Applicant: Jay Michael Pensler, Chicago, IL (US)

(72) Inventor: Jay Michael Pensler, Chicago, IL (US)

(21) Appl. No.: 14/656,707

(22) Filed: Mar. 12, 2015

Related U.S. Application Data
(60) Provisional application No. 61/953,526, filed on Mar. 14, 2014.

(51) Int. Cl. A61F 13/02; A61L 15/24; A61L 15/42; A61L 15/58

(52) U.S. Cl. CPC A61F 13/025; A61F 13/0269; A61L 15/58; A61L 15/24; A61L 15/425; A61F 2013/0028

(57) ABSTRACT

A system and method of protecting a wound on the skin that results in a reduction of scarring at the wound site. A segment of porous adhesive tape is provided. A plurality of apertures are formed into the porous adhesive tape, creating a mesh pattern. The segment of porous adhesive tape is applied to the skin using the tacky adhesive on the adhesive tape. The wound is positioned under the mesh pattern. The segment of porous adhesive tape is applied to the skin in a stretched condition. As such, the porous adhesive tape biases the skin into compression under the porous adhesive tape. The segment of porous adhesive tape and the skin under the apertures are coated with a skin adhesive. The skin adhesive bonds the segment of porous adhesive tape to the skin. This isolates the skin surrounding the wound and minimizes the stresses.

### RELATED APPLICATIONS

This application claims priority of Provisional Patent Application No. 61/953,526, filed Mar. 14, 2014.

### BACKGROUND OF THE INVENTION

1. Field of the Invention

In general, the present invention relates to systems and methods that are used to close and protect skin wounds in a manner that reduces scarring. More particularly, the present invention relates to wound closure systems and methods that use elastic elements to bias a wound closed as it heals in conjunction with an adhesive.

2. Prior Art Description

When the skin is lacerated or otherwise cut, it is well known that the wound heals best when the skin from the opposite sides of the wound are neatly brought together. The wound may have a dressing placed. The purpose of a dressing may accomplish any number of functions including isolation of the wound, compression of the wound, absorption of secretions from the wound, immobilization of the wound or enhance the cosmetic appearance of the wound. The dressing can facilitate wound healing and, therefore, minimizes the size of any scar that may develop. For deep wounds, the skin is often brought together with stitches, staples and/or skin glue. For less severe wounds, skin is often brought together using an adhesive bandage, such as a butterfly bandage. When using an adhesive bandage, the skin around the wound is pushed as far toward closed as is permitted by the wound prior to the application of the adhesive bandage. Once the adhesive bandage is applied, the adhesive bandage attempts to hold the wound closed as it heals. However, the bandage is rarely properly sized for the wound. As such, only certain areas of the wound are held together well by the adhesive bandage, while other areas are not.

Scarring at a wound site is often a function of a number of factors including how neatly the site of a wound can be set together as the wound begins to heal, the tension on the wound closure and the length of time the tension is applied to the wound. Neat cuts can heal without significant scarring. Cuts that are jagged or are stressed open during the healing process tend to scar. Wounds that are stitched, stapled or glued together are not protected from stresses for extended periods of time. As a person moves, different areas of that person's skin experience both tension stresses and compression stresses. If tension stresses are experienced at the site of the wound, the tension stresses can pull the wound. Although the stitches, staples, and/or glue may remain intact, the tension stresses disrupt the wound and alter the new scar being regenerated at the wound site. The result is that the skin being regenerated and or healed develops significant scar tissue. More importantly, extended stress on the wound as occurs with excision of tissue and wound closure or multiple additional scenarios during surgery often result in a widened or hypertrophic scar. This can produce a life-long scar on the skin which is wide, noticeable and unsightly.

The use of sutures and skin undermining prior to closure can reduce the amount of tension stresses experienced at a wound site. However, stresses still reach the wound. The wound typically is stressed in a manner to result in opening (dehiscence) even though the adhesive bandage is in place. Furthermore, adhesive bandages are temporary and are not intended to be placed over a wound indefinitely. Rather, adhesive bandages are designed to be in place for a very short period of time. Adhesive bandages are also designed to firmly stick to the skin. As such, when a bandage is pulled from the skin, it inevitably pulls the skin in tension. This can stress a wound and increase the tendency of the wound to reopen and generate scar tissue. It can also aggravate the skin and create conditions near the wound that are prone to infection.

In the prior art, adhesive bandages have been developed that are intended to reduce the amount of scarring at a wound site. Such adhesive bandages completely surround the wound and try to prevent the skin around the wound from experiencing any significant stresses. Such prior art bandages are exemplified by U.S. Pat. No. 7,683,234 to Gurtner. Although such adhesive bandages do protect a wound site to some degree, the protection is often inadequate. The adhesive bandage must be changed frequently. The adhesive bandages also must be removed and replaced for bathing or if left in place to bath the dressing does not provide isolation of the wound. Each time the adhesive bandage is removed, it irritates the skin around the wound and pulls at the skin adjacent the wound. Accordingly, adhesive bandages are poor choices for deep wounds that may require many days to heal and numerous bandage changes during the healing period. Furthermore, adhesive bandages often become loose in a short period of time, especially in areas of the body, such as the head, abdomen, and torso, where the bandage cannot be wrapped onto itself. As such, adhesive bandages do not work well on deep wounds that are caused by many surgical incisions, especially to surgical incisions on the head, chest, or abdomen.

A need therefore exists for a wound closure system that better minimizes stresses to the skin surrounding a wound. A need also exists for a wound closure system that does not require periodic changing and can be used for deep surgical incisions on the head and/or torso of the body. A need exists for a system to provide improved wound isolation from external contaminants. A need exists for a system to provide extended reduction in skin tension while wound healing occurs. These needs are met by the present invention as described and claimed below.

### SUMMARY OF THE INVENTION

The present invention is a system and method of protecting a wound on the skin that results in an improved ability for the wound to heal favorably with significant prolonged tension reduction of the wound in conjunction with superior isolation, immobilization and compression of the wound. A segment of porous elastic adhesive tape is provided. Pluralities of apertures are formed into the porous adhesive tape, creating a mesh pattern. The segment of porous adhesive tape is stretched and then applied to the skin using the tacky adhesive on the adhesive tape. The wound is positioned under the mesh pattern. The segment of porous adhesive tape is applied to the skin in a stretched condition at 90 degrees to the wound edge. As such, the porous elastic adhesive tape biases the skin into compression under the porous adhesive tape.

The segment of porous adhesive tape and the skin under the apertures are coated with a skin adhesive, such as cyanoacrylate. The skin adhesive bonds the segment of porous adhesive tape to the skin in a compressed state. This application of the dressing isolates the skin surrounding the wound and the wound itself and reduces the tension on the wound and stresses that affect the wound during healing. This enables the wound to heal without developing as much scar tissue. In addition, the adhesive over the tape reinforces the wound and provides significantly increased bursting strength to the wound after closure.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 shows a wound and a segment of skin;

FIG. 2 shows the components contained within an exemplary embodiment of the present invention wound care system;

FIG. 3 shows a side view of the exemplary embodiment of the wound care system in conjunction with a skin wound;

FIG. 4 shows the embodiment of FIG. 3 with the wound care system applied to the skin over a wound; and

FIG. 5 shows the embodiment of FIG. 4 from a top view.

### DETAILED DESCRIPTION OF THE DRAWINGS

Although the present invention wound care system and method can be used to protect many different types of wounds on a person's skin, the wound care system is particularly well suited for protecting surgical incision wounds, which are typically deep wounds with clean, smooth edges. The embodiment illustrated shows the wound care system being used to close a long surgical incision. This embodiment is selected for the purposes of description and explanation. The illustrated embodiment, however, is merely exemplary and should not be considered a limitation when interpreting the scope of the appended claims.

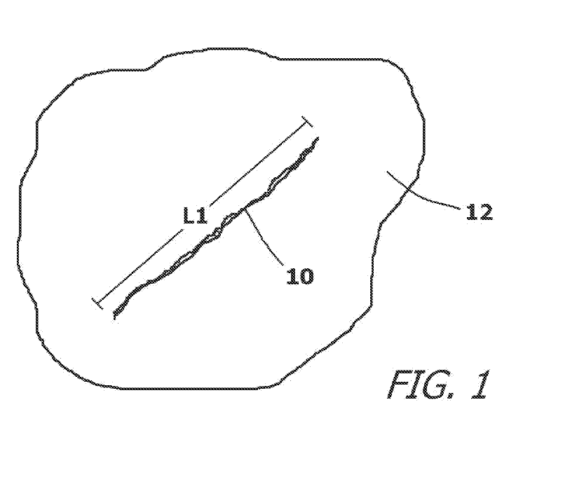

Referring to FIG. 1, a wound 10 is shown on the skin 12. The wound 10 is a result of a surgical incision, wherein the wound 10 has a length L1 that may vary from a few millimeters to several centimeters. It is the purpose of the present invention to provide a system and method of protecting the wound 10 in such a manner that it minimizes scarring at the wound site, minimizes the chances of infection, and minimizes the occurrence of reinjury to the wound 10 as it heals.

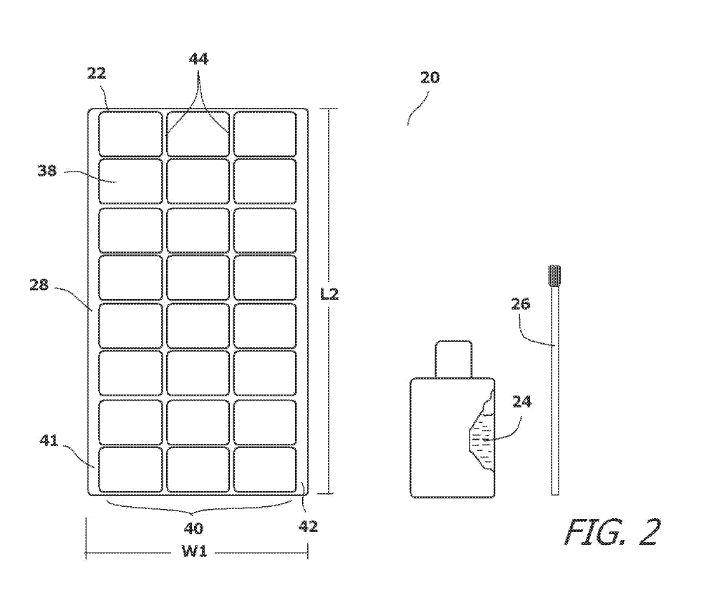

Referring to FIG. 2, the wound care system 20 is shown. The wound care system 20 includes a mesh assembly 22, a skin adhesive 24, and an applicator 26. The mesh assembly 22 includes a length L2 of medical tape 28. A preferred medical tape 28 has a tape thickness of between 8 mils and 16 mils. The medical tape 28 has elastic properties that enable the tape to elastically stretch when stressed and return to size when unstressed. The medical tape 28 is porous and is provided in a sterile condition.

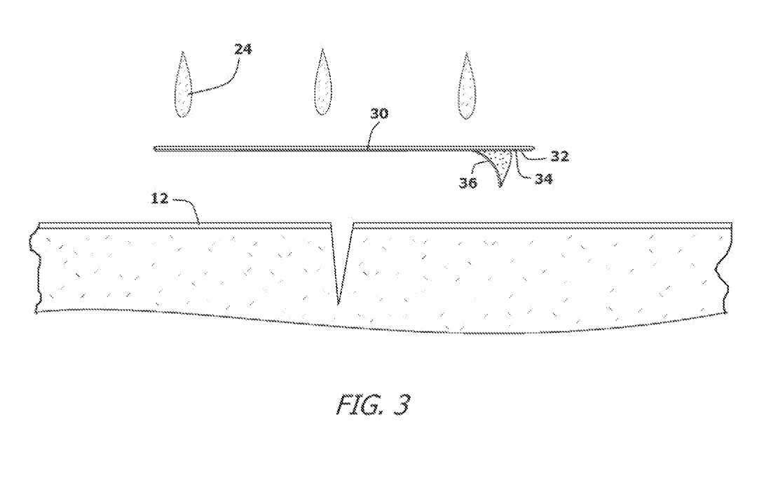

Referring to FIG. 3 in conjunction with FIG. 2, it can be seen that the medical tape 28 has a front surface 30 and an opposite rear surface 32. The rear surface 32 is coated with a hypoallergenic tacky adhesive 34. The tacky adhesive 34 is protected by a peel-away liner 36. An appropriate medical tape 28 is the hi-tack medical tape sold by 3M corporation as product number 3M-9904.

The mesh assembly 22 is preformed by the manufacturer into a mesh pattern. Apertures 38 are formed through the medical tape 28 and the tacky adhesive 34. In the shown embodiment, the apertures 38 are square in shape. However, it will be understood that other aperture shapes, such as rectangles, circles, and triangles can also be used. The apertures 38 are cut through a central section 40 of the medical tape 28. This leaves solid side borders 41, 42 that are uncut. The borders 41, 42 run the length of the medical tape 28. This maintains the integrity of the medical tape 28 along its long edges and prevents the medical tape 28 from accidentally tearing while being manipulated and applied to the skin 12.

The medical tape 28 has a width W1. The side borders 41, 42 have a combined width of between 1% and 25% of that width W1. The apertures 38 may have a combined width of between 50% and 97% of the width W1. Accordingly, the muntins 44 between the apertures 38 are thin and have a combined width of fewer than 10% of the width W1.

The length L2 of the mesh assembly 22 is selected by the healthcare provider so as to provide proper coverage over the wound 10. The wound care system 20 includes a volume of a skin adhesive 24, such as cyanoacrylate. The skin adhesive 24 can be supplied in the form of a liquid or a gel. An applicator 26 is provided that is capable of applying the skin adhesive 24 to the skin 12. In the shown embodiment, the applicator 26 is a small brush. However, other applicators, such as a pump spray applicator or a roller applicator can be used. Preferably, a single coating of skin adhesive 24 is applied.

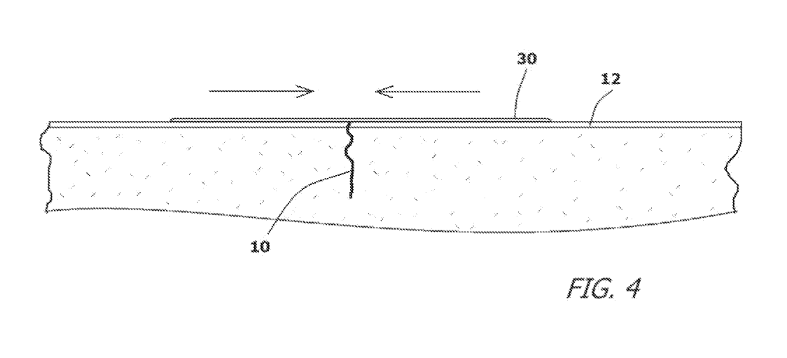

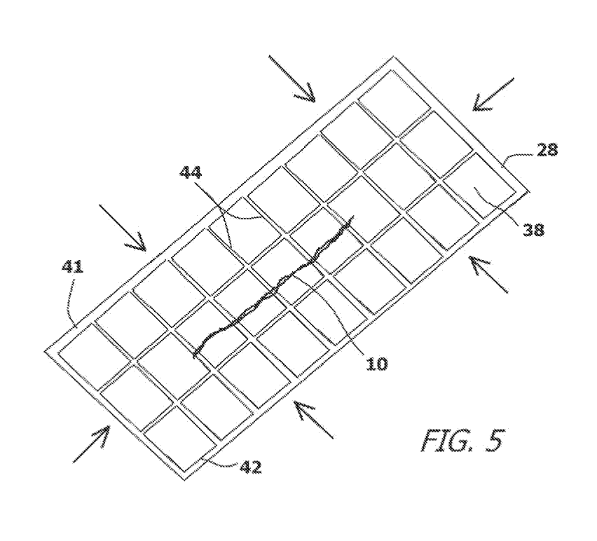

Referring to FIG. 3, FIG. 4, and FIG. 5 in conjunction with FIG. 1 and FIG. 2, it will be understood that in order to utilize the wound care system 20, the wound 10 is prepared for bandaging. The skin 12 surrounding the wound 10 is cleaned. The wound 10 is opposed. Depending upon the size of the wound 10, the wound 10 may be bound closed using stitches, staples and/or skin adhesive. A length of the mesh assembly 22 is selected. The selected length is preferably long enough to cover and isolate the wound 10. However, shorter lengths can be used. The peel-away liner 36 is removed from the mesh assembly 22, exposing the tacky adhesive 34 on the medical tape 28. The medical tape 28 is slightly stretched along its width. The medical tape 28 is then applied to the skin 12 over the wound 10 in its slightly stretched condition. Since the medical tape is slightly stretched and is elastic, it will contract once applied. This applies a compression force to the skin 12 surrounding the wound 10 that biases the skin 12 in compression toward the wound 10. The compression forces bias the wound 10 closed, as shown by the arrows in FIG. 5.

Once the medical tape 28 is applied to the skin 12, the medical tape 28, the exposed skin 12 in the apertures 38, and the area immediately surrounding the medical tape 28 are coated with the skin adhesive 24. The skin adhesive 24 saturates the porous medical tape 28 and bonds the medical tape 28 to the skin 12. The skin adhesive 24 also seals the medical tape 28 and the skin 12, therein protecting the medical tape 28 and the skin 12 from external contaminants.

The bond made by the skin adhesive 24 is permanent and lasts until the surface layer of skin 12 naturally sheds from the body. This typically takes between 10 and 20 days. The result is that the medical tape 28 is bonded in place while applying a slight compression to skin 12 surrounding the wound 10. The skin 12 surrounding the wound 10 under the medical tape 28 is immobilized by the skin adhesive 24 and the adhesive coated medical tape 28. The area of immobilized skin 12 prevents most all tensile forces from reaching the wound 10. The immobilized area is also isolated from contaminants from the surrounding skin and the environment. As a result, the chances that the wound 10 will be stressed open and/or externally contaminated are greatly reduced.

Since the skin 12 at the wound 10 is biased together while reducing tension and bacterial exposure on the wound, the wound 10 can heal with the smallest amount of scar tissue being generated. This minimizes and often eliminates any visible scarring at the wound site. The skin adhesive further coats the skin in the open apertures 38. Adhesives, such as medical grade cyanoacrylates, effectively isolate and protect the wound from surrounding contaminants.

The large apertures 38 in the medical tape 28 serve multiple purposes. First, they enable the wound 10 and the skin 12 around the wound 10 to be observed for signs of infection or other complications as the wound 10 heals. Second, they minimize the amount of medical tape 28 that passes directly over the wound 10. Only a few of the muntins 44 typically cross the wound 10. This minimizes any adherence to the actual wound 10 that could pull at the wound 10 should the medical tape 28 be inadvertently snagged during healing. Third, the minimal contact between the actual wound 10 and the medical tape 28 minimizes the areas where bacteria can harbor. The thin muntins 44 of the mesh pattern are easily saturated with the skin adhesive 24 as the skin adhesive 24 is applied. This renders the muntins 44 inert since the pours of the medical tape 28 are filled with skin adhesive 24 and cannot absorb any discharge from the wound 10.

The larger side borders 41, 42 of the medical tape 28 do not pass over the wound 10. As the wound 10 heals and the skin 12 naturally begins to shed (undergoes deepithelialization) from the body, the medical tape 28 will become loose. The medical tape 28 can be removed by grasping a section of a side border 41, 42 and pulling the medical tape 28 free of the body. If the wound site is sensitive, the medical tape 28 can be wiped with a solvent, such as acetone, that dissolves the skin adhesive 24.

It will be understood that the embodiment of the present invention that is illustrated and described is merely exemplary and that a person skilled in the art can make many variations to that embodiment. For instance, the mesh assembly can be manufactured into different sizes to accommodate wounds of different sizes and shapes. All such embodiments are intended to be included within the scope of the present invention as defined by the claims.

What is claimed is:

1. A method of protecting a wound on the skin, said method comprising the steps of:
providing a segment of porous adhesive tape having a plurality of apertures cut therein;
providing a volume of a skin adhesive;
adhering said segment of porous adhesive tape to the skin, wherein at least some of said plurality of apertures extend over the wound;
coating said segment of porous adhesive tape and the skin under said plurality of apertures with said skin adhesive, wherein said skin adhesive bonds said segment of porous adhesive tape to the skin.

2. The method according to claim 1, wherein said segment of porous adhesive tape has elastic properties and said method further includes the step of stretching said segment of porous adhesive tape when adhering said segment of porous adhesive tape to the skin, wherein said segment of porous adhesive tape compresses the skin toward the wound.

3. The method according to claim 1, wherein said step of coating said segment of porous adhesive tape includes saturating said segment of porous adhesive tape with said skin adhesive.

4. The method according to claim 1, wherein said segment of porous adhesive tape has a front surface, a rear surface, and two long edges that are separated by a width, wherein said front surface and said rear surface extend across said width between said two long edges.

5. The method according to claim 4, wherein said step of providing a segment of porous adhesive tape includes providing a segment of porous tape having solid borders that run along said two long edges, wherein said plurality of apertures are disposed between said solid borders.

6. The method according to claim 4, wherein said solid borders occupy between 1% and 25% of said width of said segment of porous adhesive tape.

7. The method according to claim 4, wherein said plurality of apertures occupy between 50% and 97% of said width of said segment of porous adhesive tape.

8. The method according to claim 1, wherein said segment of porous adhesive tape has a tape thickness of between 8 mils and 16 mils.

9. The method according to claim 4, wherein said segment of porous adhesive tape has a coating of a tacky adhesive on said rear surface.

10. The method according to claim 9, further including the step of providing a peel-away liner over said coating, wherein said peel-away liner is removed prior to said segment of porous adhesive tape being applied over the wound.

11. The method according to claim 1, wherein said skin adhesive is cyanoacrylate.

12. A method of protecting a wound on the skin, said method comprising the steps of:
providing a segment of porous elastic tape having a plurality of apertures cut therein;
providing a volume of a skin adhesive;
stretching said segment of porous elastic tape into a stretched condition;
adhering said segment of porous elastic tape to the skin in said stretched condition, wherein at least some of said plurality of apertures extend over the wound and wherein said segment of porous elastic tape compresses said wound.

13. The method according to claim 12, wherein said step of adhering said segment of porous elastic tape to the skin includes providing a first adhesive on said segment of porous elastic tape prior to said segment of porous elastic tape being applied over the wound.

14. The method according to claim 13, wherein said step of adhering said segment of porous elastic tape to the skin includes coating said segment of porous elastic tape and the skin under said plurality of apertures with said skin adhesive, wherein said skin adhesive bonds said segment of porous elastic tape to the skin.

15. The method according to claim 14, wherein said step of coating said segment of porous elastic tape includes saturating said segment of porous elastic tape with said skin adhesive.

16. The method according to claim 12, wherein said segment of porous elastic tape has a front surface, a rear surface, and two long edges that are separated by a width, wherein said front surface and said rear surface extend across said width between said two long edges.

17. The method according to claim 16, wherein said step of providing a segment of porous elastic tape includes providing a segment of porous elastic tape having solid borders that run along said two long edges, wherein said plurality of apertures are disposed between said solid borders.

18. The method according to claim 17, wherein said solid borders occupy between 1% and 25% of said width of said segment of porous elastic tape.

19. The method according to claim 17, wherein said plurality of apertures occupy between 50% and 97% of said width of said segment of porous elastic tape.

20. The method according to claim 12, wherein said segment of tape has a tape thickness of between 8 mils and 16 mils.
